SEQUENCE LISTING (21 sequences)

SEQ ID NO: 1 - DNA, 13 bp, Dependoparvovirus sp.
  source 1..13: mol_type = genomic DNA
ctccattttgaag

SEQ ID NO: 2 - DNA, 13 bp, synthetic construct
  source 1..13: mol_type = other DNA
ctccctttggaag

SEQ ID NO: 3 - DNA, 13 bp, synthetic construct
  source 1..13: mol_type = other DNA
ctccgctttgaag

SEQ ID NO: 4 - DNA, 13 bp, synthetic construct
  source 1..13: mol_type = other DNA
ctccgttttgaag

SEQ ID NO: 5 - DNA, 13 bp, synthetic construct
  source 1..13: mol_type = other DNA
ctcctttttgaag

SEQ ID NO: 6 - DNA, 13 bp, synthetic construct
  source 1..13: mol_type = other DNA
ctccaatttgaag

SEQ ID NO: 7 - DNA, 13 bp, synthetic construct
  source 1..13: mol_type = other DNA
ctcccatttgaag

SEQ ID NO: 8 - DNA, 38 bp, Dependoparvovirus sp.
  source 1..38: mol_type = genomic DNA
gcccgagtgagcacgcagggtctccattttgaagcgga

SEQ ID NO: 9 - DNA, 21 bp, synthetic construct
  source 1..21: mol_type = other DNA
gcccgagtgagcacgcagggt

SEQ ID NO: 10 - DNA, 17 bp, synthetic construct
  source 1..17: mol_type = other DNA
ctccattttgaagcgga

SEQ ID NO: 11 - DNA, 38 bp, synthetic construct
  source 1..38: mol_type = other DNA
gcccgagtgagcacgcagggtctccctttggaagcgga

SEQ ID NO: 12 - DNA, 162 bp, Dependoparvovirus sp.
  source 1..162: mol_type = genomic DNA
gaggggtggagtcgtgacgtgaattacgtcatagggttagggaggtcctgtattagaggtcacgtgagtgttttgcgacattttgcgacaccatgtggtcacgctgggtatttaagcccgagtgagcacgcagggtctccattttgaagcgggaggtttgaa

SEQ ID NO: 13 - DNA, 190 bp, Dependoparvovirus sp.
  source 1..190: mol_type = genomic DNA
cgcgaagcgcctcccacgctgccgcgtcagcgctgacgtaaattacgtcataggggagtggtcctgtattagctgtcacgtgagtgcttttgcgacattttgcgacaccacgtggccatttagggtatatatggccgagtgagcgagcaggatctccattttgaccgcgaaatttgaacgagcagcagcc

SEQ ID NO: 14 - DNA, 224 bp, Dependoparvovirus sp.
  source 1..224: mol_type = genomic DNA
ctgacgtcaatgtgacgtcctagggttagggaggtccctgtattagcagtcacgtgagtgtcgtatttcgcggagcgtagcggagcgcataccaagctgccacgtcacagccacgtggtccgtttgcgacagtttgcgacaccatgtggtcaggagggtatataaccgcgagtgagccagcgaggagctccattttgcccgcgaattttgaacgagcagcagcc

SEQ ID NO: 15 - DNA, 191 bp, Dependoparvovirus sp.
  source 1..191: mol_type = genomic DNA
gcgcgaagcgcctcccacgctgccgcgtcagcgctgacgtaaattacgtcataggggagtggtcctgtattagctgtcacgtgagtgcttttgcggcattttgcgacaccacgtggccatttgaggtatatatggccgagtgagcgagcaggatctccattttgaccgcgaaatttgaacgagcagcagcc

SEQ ID NO: 16 - DNA, 137 bp, synthetic construct
  source 1..137: mol_type = other DNA
catagaggtcctgtattagaggtcacgtgagtgttttgcgacagtttgcgacaccatgtggtcacaagaggtatataaccgcgagtgagccagcgaggagctccattttgcccgcgaagtttgaacgagcagcagcc

SEQ ID NO: 17 - DNA, 137 bp, synthetic construct
  source 1..137: mol_type = other DNA
catagaggtcctgtattagaggtcacgtgagtgttttgcgacattttgcgacaccatgtggtcacgctgggtatttaaccgcgagtgagcgaaccaggagctccattttgcccgcgaattttgaacgagcagcagcc

SEQ ID NO: 18 - DNA, 13 bp, synthetic construct
  source 1..13: mol_type = other DNA
  variation 5..6: note = nn denotes any dinucleotide except at
ctccnntttgaag

SEQ ID NO: 19 - DNA, 13 bp, synthetic construct
  source 1..13: mol_type = other DNA
  variation 9: note = n may be an a, c, t or g, with the proviso that when n at position is a, n at postion 9 cannot be t
ctccntttngaag

SEQ ID NO: 20 - DNA, 24 bp, synthetic construct
  source 1..24: mol_type = other DNA
  misc_feature 1: note = 3'-3' phosphodiester bond to a thymidine
  misc_feature 24: note = 3'-3' phosphodiester bond to a thymidine
gctcgctcgctcgctcgctcgctc

SEQ ID NO: 21 - DNA, 24 bp, synthetic construct
  source 1..24: mol_type = other DNA
  misc_feature 1: note = 3'-3' phosphodiester bond to a thymidine
  misc_feature 24: note = 3'-3' phosphodiester bond to a thymidine
gagcgagcgagcgagcgagcgagc

(19) United States
(12) Patent Application Publication
(10) Pub. No.: US 2026/0297617 A1
(43) Pub. Date: Oct. 1, 2026

(54) Modified AAV P5 Promoter for Improved Vector Titer and Purity

(71) Applicant: St. Jude Children's Research Hospital, Inc., Memphis, TN (US)

(72) Inventors: Stephen Winston, Memphis, TN (US); Mark Brimble, Memphis, TN (US); Isaiah Reeves, Memphis, TN (US); Andrew Davidoff, Memphis, TN (US)

(21) Appl. No.: 19/477,834

(22) PCT Filed: Apr. 29, 2024
(86) PCT No.: PCT/US2024/026742

§ 371 Date: Oct. 23, 2025

Related U.S. Application Data
(60) Provisional application No. 63/499,297, filed on May 1, 2023.

(51) Int. Cl. C12N 15/86; C07K 14/005

(52) U.S. Cl. CPC C12N 15/86; C07K 14/005; C12N 2750/14122; C12N 2750/14143

(57) ABSTRACT

A recombinant AAV vector including an AAV2 P5 homologue or modified REP nicking site is described, wherein the recombinant AAV vector provides for a reduction in DNA contamination upstream of the PS promoter and improved purity.

### INTRODUCTION

This application claims benefit from U.S. Provisional Patent Application Ser. No. 63/499,297, filed May 1, 2023, the content of which is incorporated herein by reference in its entirety.

### STATEMENT REGARDING ELECTRONIC FILING OF A SEQUENCE LISTING

A Sequence Listing in XML text format, submitted under 37 C.F.R. § 1.831-1.834, entitled “SJ0110WO_ST26.XML,” 22,796 bytes in size, generated Apr. 13, 2024, is provided in lieu of a paper copy. This Sequence Listing is hereby incorporated by reference into the specification.

### BACKGROUND

The relative ease and versatility of recombinant adeno-associated virus (rAAV) production, coupled with its robust transduction of non-dividing cells, has made AAV a popular gene delivery tool. Clinical successes in the treatment of hemophilia and other monogenic disorders have helped to increase interest in AAV-mediated gene transfer as a treatment strategy. Although rAAV therapies are providing significant clinical benefit, improving their safety remains a top priority. Post-treatment immunogenic responses have been observed in several trials and in certain research models dose-dependent toxicity has been reported.

The wild type AAV genome is composed of four overlapping same-frame replication (Rep) genes and three overlapping same-frame capsid (Cap) genes. Additional alternate reading frame proteins membrane-associated accessory protein and AAV assembly protein exist embedded within the capsid gene region. Expression of these proteins is driven by three promoters: P5, P19, and P40. P5 drives the large REP proteins REP78 and REP68, P19 drives the short REP proteins REP52 and REP40, and P40 drives expression of the capsid genes VP1, VP2 and VP3. This 4.7 kb genome is flanked by inverted terminal repeats (ITRs). In rAAV production, the ITRs flank an expression cassette of interest that is packaged into the rAAV virion. The replication and capsid genes are, in most cases, delivered on a separate plasmid construct. The AAV2 P5 promoter is used in most clinical human embryonic kidney 293T-based rAAV production systems. As in wild type AAV, P5 transcribes REP78 and REP68 in rAAV production. P5 can achieve this positioned either in its wild type location relative to Rep or directly downstream of the capsid gene, with promoter activity reading through the plasmid backbone. qPCR analysis of rAAV preparations has demonstrated the presence of contaminant sequences from upstream of P5 on the REP-CAP production plasmid packaged within rAAV virions raising safety concerns.

Previous efforts to remove these contaminants have focused on design interventions such as increasing the size of the expression cassette backbone plasmid and the implementation of DNA minicircles to the production process. However, there has been comparatively little investigation into the cause and effects of these DNA impurities.

While replacing the P5 promoter with the HIV-LTR promoter or a combination of the CMV promoter and metallothionein promoter has been suggested (see U.S. Pat. No. 5,658,776 and Allen et al. (2000) Mol. Ther. 1(1):88-95), there was no indication of a change in the level of DNA contamination. Likewise, while the insertion of a spacer located between the 3′ end of the AAV P5 promoter and the 5′ end of the rep gene start codon has been shown to modulate REP protein expression (see WO 2019/217483), there was no indication of a change in the level of DNA contamination.

WO 2021/242664 describes a reduction in DNA contamination by inserting an exogenous spacer between the REP binding site and a transcription start site-localized Ying-Yang 1 (YY1) binding site in the P5 promoter. However, there remains a need in the art for alternative AAV vectors that prevent packaging of unintended nucleic acids during rAAV production thereby improving purity and safety.

### SUMMARY OF THE INVENTION

This invention provides a recombinant adeno-associated virus (AAV) vector harboring a polynucleotide encoding AAV2 Replication (REP) 68/78 proteins operably linked to (a) a P5 promoter homologue; (b) a P5 promoter including a terminal resolution sequence having a nucleotide sequence of ctccnntttgaag (SEQ ID NO:18), wherein “nn” may be any dinucleotide except “at;” or (c) a P5 promoter including a terminal resolution sequence having a nucleotide sequence of ctccn₁tttn₂gaag (SEQ ID NO:19), wherein n₁ and n₂ may each independently be an a, c, t or g, with the proviso that when n₁ is “a,” n₂ is not “t.”

This invention also provides a recombinant P5 promoter including a terminal resolution sequence having a nucleotide sequence of ctccnntttgaag (SEQ ID NO:18), wherein “nn” may be any dinucleotide except “at;” or a nucleotide sequence of ctccn₁tttn₂gaag (SEQ ID NO:19), wherein n₁ and n₂ may each independently be an a, c, t or g, with the proviso that when n₁ is “a,” n₂ is not “t.”

A host cell including an AAV vector herein is also provided as is a method increasing titer and/or purity of a recombinant adeno-associated virus (rAAV).

### BRIEF DESCRIPTION OF THE DRAWINGS

FIG. 1 depicts the structure of the AAV2 P5 promoter, an AAV2 P5 promoter with an exogenous spacer between the REP binding site and a YY1 binding site (P5-HS system), and an AAV2 P5 promoter with a terminal resolution sequence (TRS) loop sequence derived from AAV5 (P5 promoter-AAV5 loop).

FIG. 2 shows levels of contaminant DNA (upper panel) and titers of FIX when the P5 promoter of a AAV2 construct (CR21) includes: a spacer between the YY1 box and the P5 REP binding site (CR21+HS5), a TRS loop sequence obtained from AAV5 (AAV2 P5+AAV5 TRS loop), or a TRS loop sequence including a GC (TRS dinucleotide GC), GT (TRS dinucleotide GT), TT (TRS dinucleotide TT), AA (TRS dinucleotide AA), or CA (TRS dinucleotide CA) dinucleotide.

FIG. 3 shows the design of a green fluorescent protein (GFP) reporter assay to quantify the impact of promoter modifications.

FIG. 4 shows that TRS alterations which significantly reduce contamination also impact promoter function.

FIG. 5 shows that changing the REP start codon from ACG to ATG improves viral titer.

FIG. 6 shows that changing the REP start codon from ACG to ATG does not alter the level of DNA contamination.

FIG. 7 shows that replacing the AAV2 P5 promoter with P5 homologues can reduce contamination outside of the P5 promoter and outside of the inverted terminal repeats (ITRs). *, P=0.0127; **, P=0.0414; ***, P=0.0376.

FIG. 8 shows that replacing the AAV2 P5 promoter with P5 homologues can improve viral titer compared to AAV2 P5. *, P=0.0332; **, P=0.0042; ***, P=0.0007.

FIG. 9 shows that the AAV13 P5 homologue (P13) does not tolerate TRS modifications such as a GC dinucleotide TRS loop (GC di), AA dinucleotide TRS loop (AA di) or the AAV5 TRS loop (AAV5 TRS).

FIG. 10 shows western blot analysis of large REP and small REP expression. Protein levels were determined in lysate at 12 hours (upper panel) and 48 hours (lower panel) post transfection.

FIG. 11 shows that DNA contamination is reduced from 2% to 0.05% in TRS-modified constructs, when vectors are prepared on a large scale.

FIGS. 12A-12B provide data showing that P5 modifications reduce promoter activity during AAV production. FIG. 12A, Schematic of construct showing that green fluorescent protein (GFP) is embedded in REP-CAP and separated with REP78 by the self-cleaving peptide p2a. FIG. 12B, Fluorescence was monitored during AAV production using an IncuCyte® Live-Cell Analysis System.

FIGS. 13A-13D show that increasing the REP-CAP plasmid ratio boosts titer but not contamination in 6-well plates. FIG. 13A, Schematics of constructs used in this analysis. FIG. 13B, Titer. FIG. 13C-13D, Contamination outside of the P5 promoter and outside of the ITRs, respectively.

FIGS. 14A-14B show that increasing REP-CAP plasmid ratio boosts titer but not contamination in 15-cm plates. FIG. 14A, Titer. FIG. 14B, Western blot analysis of REP expression.

FIGS. 15A-15C show the impact on titer of vector generated with a modified p5 promoter (FIG. 15A) and contamination (FIGS. 15B-15C) on ssAAV8 HLP vector production when including a transgene (FVIIIv3). FIG. 15A, Titer of modified P5 was about 75% of wild-type P5 in 10-layer cell stacks. FIGS. 15B-15C, Purity was maintained using 1× (FIG. 15B) or 3× (FIG. 15C) REP-CAP plasmid with a modified P5.

FIGS. 16A-16C show improvement in purity is due to competitive inhibition. AAV production plasmid was mixed with an annealed modified oligonucleotide (containing the sequence of an RBE) at 5%, 10%, 25%, 50% and 100% molar ratio to the amount of REP-CAP plasmid. Analysis of pellets at day 2 is shown. FIG. 16A, FIX Titer. FIG. 16B, P5 signal. FIG. 16C, vector purity.

FIG. 17 shows in vivo expression demonstrating that the vector produced from REP-CAPs with the modified p5 promoters had equivalent transgene expression.

### DETAILED DESCRIPTION OF THE INVENTION

Provided herein is the use of P5 homologues derived from AAV serotypes other than AAV2 or direct modification of the REP nicking site of the AAV2 P5 promoter to eliminate active contaminant incorporation from a REP-CAP plasmid, while maintaining or improving rAAV titers. Accordingly, an AAV vector (e.g., a rAAV vector or plasmid) harboring a P5 promoter homologue or a P5 promoter including a modified REP nicking site is provided a is the use of the same in a method for increasing rAAV titer and/or purity. The AAV vector and method described herein find application in the production of rAAV, e.g., via REP-CAP plasmids, in the commercial and/or research setting, as well as in some recombinant bocaviral production systems using AAV REP proteins.

As used herein, the term “vector” refers to a polynucleotide capable of transporting another polynucleotide to which it has been linked. Vectors include, but are not limited to, polynucleotide molecules that are single-stranded double-stranded, or partially double-stranded; polynucleotide molecules that include one or more free ends, no free ends (e.g., circular); polynucleotide molecules that include DNA, RNA, or both; and other varieties of polynucleotides known in the art. One type of vector is a “plasmid,” which refers to a circular double stranded DNA loop into which additional DNA segments can be inserted, such as by standard molecular cloning techniques. Another type of vector is a viral vector, wherein virally-derived DNA or RNA sequences are present in the vector for packaging into a virus (e.g., an adeno-associated virus). Viral vectors also include polynucleotides carried by a virus for transduction into a host cell.

A “recombinant” nucleic acid or polynucleotide refers to a nucleotide sequence that is produced by means of molecular biological techniques (e.g., cloning, enzyme restriction and/or ligation steps) and/or chemical synthesis.

The terms “adeno-associated virus” or “AAV,” “recombinant AAV” or “rAAV,” unless specified, include serotypes AAV), AAV2, AAV3 (including types 3A and 3B), AAV4, AAV5, AAV6, AAV7, AAV8, AAV9, AAV10, AAV11, AAV12, AAV13, avian AAV, bovine AAV, canine AAV, equine AAV, ovine AAV and recombinantly generated capsids prepared, e.g., by directed evolution, peptide display and the like. Typically, AAV can infect both dividing and non-dividing cells and can be present in an extrachromosomal state without integrating into the genome of a host cell. AAV also includes codon-optimized AAV.

As used herein, “polynucleotide encoding AAV2 Replication (REP) 68/78 proteins” refers to the polynucleotide that encodes the REP78 and REP68 proteins respectively produced from unspliced and spliced transcripts from the P5 promoter. REP78 and REP68 are multifunctional proteins with largely overlapping functions in almost every stage of the AAV life cycle, such as site-specific DNA binding, helicase activity, and/or site-specific endonuclease activity. REP78 and/or REP68 are required in trans for AAV replication and/or excision from the host genome (U.S. Pat. Nos. 5,658,776; 5,837,484; WO 98/27207; U.S. Pat. Nos. 5,658,785; 7,785,888). Polynucleotides encoding AAV2 REP68/78 proteins of use in herein are well known in the art. For example, the genomic sequences of AAV2 are provided in GENBANK accession No. J01901, AF043303, and NC 001401.2.

As is known in the art, the AAV2 P5 promoter is a regulatory element that controls the expression of the AAV2 REP68 and REP78 proteins. In the absence of a helper virus, P5 is repressed by YY1 and has very minimal activity. In the presence of a helper virus, expression is increased, and REP is produced. Low levels of REP expression repress transcription from the P5 promoter. REP-mediated repression is attributed to REP binding to a REP binding element (RBE) located between the TATA and YY1+1 site of the P5 promoter. Located downstream of the P5 RBE is a terminal resolution sequence (ctccattttgaag; SEQ ID NO:1) sequence positioned within the YY1+1 site of the P5 element. When combined with the RBE and the TATA element, the P5 TRS can undergo REP-dependent nicking and DNA amplification. Not wishing to be bound by theory, it is believed that using a P5 homologue or modifying the REP nicking site of the AAV2 P5 promoter allows the REP proteins to efficiently bind to, but not nick the DNA. This enables the maintenance of the autoregulation of REP68/78 expression, albeit at a lower overall level. Therefore, efficient vector production is maintained, while interfering with unintended replication initiating outside of the P5 promoter.

Accordingly, in one aspect, a recombinant AAV vector is provided, wherein the wild type AAV2 P5 promoter is replaced with a P5 promoter homologue. “Wild type,” “wt” or “wild-type,” when in reference to a sequence (e.g., that is introduced into a cell and/or virus), refers to the sequence as it occurs in nature (e.g., in the cell and/or virus). Given that most or all genetic loci exist in a variety of allelic forms, which vary in frequency throughout the geographic range of a species, a “wild type” sequence may refer to the sequence that occurs at the highest frequency in nature. By way of illustration, the wild type AAV2 P5 promoter may have the nucleotide sequence of:

(SEQ ID NO: 12)
gaggggtggagtcgtgacgtgaattacgtcatagggttagggaggtcctgt
attagaggtcacgtgagtgttttgcgacattttgcgacaccatgtggtcac
gctgggtatttaagcccgagtgagcacgcagggtctccattttgaagcggg
aggtttgaa

(see, e.g., Pereira et al. (1997) J. Virol. 71(2):1079-88).

A promoter herein may encompass at least about 40, 50, 60, 70, 80, 90, 100, 110, 120, 130, 140, 150, or 160 nucleotides adjacent to (or upstream of) the transcription start site of the large REP proteins (i.e., REP78 and REP68) in an AAV genome, or a sequence having at least 90%, 91%, 92%, 93%, 95%, 96%, 97%, 98% or more sequence identity thereto.

A “P5 promoter homologue” or “AAV2 P5 promoter homologue” refers to a promoter region derived from an AAV serotype other than AAV2 that functions in a manner similar to the AAV2 P5 promoter. In some aspects, the P5 promoter homolog is derived, obtained or isolated from serotype AAV1, AAV3 (including types 3A and 3B), AAV4, AAV5, AAV6, AAV7, AAV8, AAV9, AAV10, AAV11, AAV12, AAV13, or synthetic variant promoters thereof. In other aspects, the P5 promoter homologue has the nucleotide sequence of SEQ ID NO:13-17.

In another aspect, a recombinant P5 promoter and AAV vector comprising the same are provided, wherein the REP nicking site of the wild type P5 promoter (e.g., the AAV2 P5 promoter) has been modified. In one aspect, the REP nicking site located in the terminal resolution sequence (TRS) is modified. In some aspects, the wild type P5 TRS sequence (e.g., ctccattttgaag (SEQ ID NO:1)), is replaced with a TRS having the nucleotide sequence of ctccctttggaag (SEQ ID NO:2), ctccgctttgaag (SEQ ID NO:3), ctccgttttgaag (SEQ ID NO:4), ctcctttttgaag (SEQ ID NO:5), ctccaatttgaag (SEQ ID NO:6), or ctcccatttgaag (SEQ ID NO:7). In other aspects, the wild type P5 TRS sequence (e.g., ctccattttgaag (SEQ ID NO:1)), is replaced with a TRS having the nucleotide sequence of ctccnntttgaag (SEQ ID NO:18), wherein the “nn” dinucleotide may be any dinucleotide except “at,” e.g., “nn” may be “aa,” “ac,” “ag,” “ca,” “cc,” “ct,” “ct,” “ta,” “tc,” “tt,” “tg,” “ga,” “gc,” “gt,” or “gg.” In some aspects, the wild type P5 TRS sequence (e.g., ctccattttgaag (SEQ ID NO:1)), is replaced with a TRS having the nucleotide sequence of ctccn₁tttn₂gaag (SEQ ID NO:19), wherein n₁ and n₂ may each independently be an a, c, t or g, with the proviso that when n₁ is “a,” n₂ cannot be “t.”

When operably linked to a polynucleotide encoding REP68/78 proteins, the modified P5 promoter or P5 promoter homologue herein provides for efficient vector production, while reducing or eliminating contaminant DNA. As used herein, the term “operably linked” refers to linking the sequences such that they perform their intended function. By way of illustration, operably linking a promoter sequence to a polynucleotide sequence of interest refers to linking the promoter sequence and the polynucleotide sequence of interest in a manner such that the promoter sequence is capable of directing the transcription of the polynucleotide sequence of interest and/or the synthesis of a polypeptide encoded by the polynucleotide sequence of interest.

In some aspects, wherein the promoter disclosed herein is operably linked to nucleic acids encoding AAV2 REP68/78 proteins, the REP68/78 start codon has the nucleotide sequence of ATG. As is known in the art, “codon” refers to a specific sequence of three adjacent nucleotides on a strand of DNA or RNA that specifies the genetic code information for synthesizing a particular amino acid. A “start codon” is the first codon of an RNA transcript. In some aspects, the start codon may be ACG or ATG.

The P5 promoter herein may be provided alone as an isolated polynucleotide or be provided as part of longer polynucleotide including other nucleotide sequences. Accordingly, some embodiments also provide isolated polynucleotides comprising a promoter described herein. As would be appreciated by those skilled in the art, the isolated polynucleotides may further contain one or more additional elements or sequences, such as any described herein or known in the art.

Some aspects also provide vectors harboring a modified P5 promoter described herein. The modified P5 promoter of the present disclosure can be included in any suitable vector to regulate or drive transcription of the large REP proteins, i.e., REP78 and REP68, or upregulate expression of the small REP proteins, i.e., REP52 and REP40. In some aspects, the vector is a viral vector. Ideally, the vector is an AAV vector.

In some aspects the AAV vector may be an episomal vector, i.e., a vector that does not integrate into the genome of a host cell. In other aspects, the AAV vector may be a vector that integrates into the host cell genome. Any vector system using an AAV2 REP and AAV2 P5 promoter may include the modified promoter described herein. For example, the P5 promoter and AAV2 REP gene feedback loop is used in recombinant bocavirus production (Yan, et al. (2013) Mol. Ther. 21:2181-2194).

In some aspects, the modified P5 promoter and AAV rep and cap genes are present in a plasmid. It is contemplated that AAV2 rep gene may be combined with cap genes from any AAV serotype, including, but not limited to, AAV1, AAV2, AAV4, AAV5, AAV6, AAV7, AAV8, or AAV9, and any synthetic variants thereof.

Vectors suitable for use in mammalian cells are widely described and well-known in the art. Those skilled in the art would appreciate that vectors herein that include a promoter described herein will also contain additional sequences and elements useful for the replication of the vector in prokaryotic and/or eukaryotic cells, selection of the vector and the expression of the sequences in a variety of host cells. For example, the vectors of the present disclosure may include a prokaryotic replicon (that is, a sequence having the ability to direct autonomous replication and maintenance of the vector extrachromosomally in a prokaryotic host cell, such as a bacterial host cell. Such replicons are well known in the art. In some aspects, the vectors may include a shuttle element that makes the vectors suitable for replication and integration in both prokaryotes and eukaryotes. In addition, vectors may also include a gene whose expression confers a detectable marker such as a drug resistance gene, which allows for selection and maintenance of the host cells. Vectors may also have a reportable marker, such as gene encoding a fluorescent or other detectable protein.

The vectors can also include transcriptional enhancers, translational signals, and transcriptional and translational termination signals. Examples of transcriptional termination signals include, but are not limited to, polyadenylation signal sequences, such as bovine growth hormone (BGH) poly(A), SV40 late poly(A), rabbit beta-globin (RBG) poly(A), thymidine kinase (TK) poly(A) sequences, and any variants thereof.

Also provided are recombinant virions, including recombinant AAV virions, produced using the viral vectors described herein. Most typically, the recombinant virions include a heterologous coding sequence operably linked to a promoter. As will be understood by those skilled in the art, in most instances, not all of the nucleotides of the viral vector will be packaged into the recombinant virus. For example, in the case of AAV vector packaging, only the ITRs and the nucleotides flanked by the ITRs, including a promoter as well as any other sequences downstream of the promoter and upstream of the 3′ ITR, such as a heterologous coding sequence, will be packaged into the recombinant AAV. The recombinant virions can be used to deliver the heterologous coding sequence to a host cell for expression in that cell.

Methods for packaging viral vectors to produce recombinant virions are well known in the art, and any such method can be used to produce recombinant virions. By way of illustration, methods for producing a recombinant AAV include introducing into a packaging cell line an AAV vector encoding a heterologous coding sequence, helper functions for generating a productive AAV infection, and AAV cap and rep genes, the latter of which includes a modified P5 promoter as describe herein; and recovering a recombinant AAV from the supernatant of the packaging cell line. Various types of cells can be used as the packaging cell line. For example, packaging cell lines that can be used include, but are not limited to, HEK 293 cells, HeLa cells, and Vero cells, for example, as disclosed in US 2011/0201088.

The helper functions may be provided by one or more helper plasmids or helper viruses harboring adenoviral helper genes. Non-limiting examples of the adenoviral helper genes include E1A, E1B, E2A, E4 and VA, which can provide helper functions to AAV packaging. Helper viruses of AAV are known in the art and include, for example, viruses from the family Adenoviridae and the family Herpesviridae. Examples of helper viruses of AAV include, but are not limited to, SAdV-13 helper virus and SAdV-13-like helper virus described in US 2011/0201088, and helper vectors pHELP (Applied Viromics). A skilled artisan will appreciate that any helper virus or helper plasmid of AAV that can provide adequate helper function to AAV can be used herein.

In some instances, recombinant AAV is produced by using a cell line that stably expresses some of the necessary components for AAV virion production. For example, a plasmid (or multiple plasmids) including the modified P5 promoter, AAV rep and cap genes, and a selectable marker, such as a neomycin resistance gene, can be integrated into the genome of a cell (the packaging cells). The packaging cell line can then be co-infected with a helper virus (e.g., adenovirus providing the helper functions) and an AAV vector for expressing a heterologous gene of interest. The advantages of this method are that the cells are selectable and are suitable for large-scale production of the recombinant AAV. As another non-limiting example, adenovirus or baculovirus rather than plasmids can be used to introduce rep and cap genes into packaging cells. As yet another non-limiting example, both the AAV vector for expressing a heterologous gene of interest and the rep-cap genes (including the modified P5 promoter) can be stably integrated into the DNA of producer cells, and the helper functions can be provided by a wild type adenovirus to produce the recombinant AAV.

As will be appreciated by a skilled artisan, any method suitable for purifying AAV can be used in the embodiments described herein to purify the recombinant AAV, and such methods are well known in the art. For example, the recombinant AAV can be isolated and purified from packaging cells and/or the supernatant of the packaging cells. In some aspects, the AAV is purified by separation method using a CsCl gradient. In other aspects, AAV is purified as described in US 2002/0136710 using a solid support that includes a matrix to which an artificial receptor or receptor-like molecule that mediates AAV attachment is immobilized.

Also provided herein are host cells harboring an AAV vector or virion of the present disclosure. In some instances, the host cells are used to amplify, replicate, package and/or purify a vector or virion. In other examples, the host cells are used to express a heterologous coding sequence under the control of a suitable promoter. In some aspects, the methods may be in vitro, ex vivo or in vivo.

Exemplary host cells include prokaryotic and eukaryotic cells. In some instances, the host cell is a mammalian host cell. In instances where the cells are used to package a viral vector described herein, the cells may also be transfected with one or more plasmids or infected with one or more viruses that provide the necessary helper and accessory molecules for packaging. In further example, the host cells may stably express, such as from the genome, one or more helper and accessory molecules. It is well within the skill of a skilled artisan to select an appropriate host cell for the amplification, replication, packaging and/or purification of a vector or recombinant virion described herein. Exemplary mammalian host cells include, but are not limited to, HEK-293 cells, HeLa cells, Vero cells, HUH7 cells, and HepG2 cells.

Also provided is a method of increasing titer and/or purity of a rAAV by operably linking nucleic acids encoding AAV2 REP68/78 proteins to a P5 promoter homologue or a P5 promoter including a TRS having a nucleotide sequence of SEQ ID NO:2-7. As demonstrated herein, P5 promoter homologues and P5 promoters including a modified REP nicking site, result in the production of higher quality clinical AAV products while maintaining or increasing titers compared to conventional production systems. In some aspects of this method, rAAV titer and/or purity is increased by about 10%, about 20%, about 30%, about 40%, about 50%, about 60%, about 70% or more as compared to a rAAV that does not include a P5 promoter homologue or modified P5 promoter described herein, e.g., an rAAV including a wild type P5 promoter. rAAV purity may be assessed as described herein by the presence/level of encapsidated DNA contaminants.

An advantage of this invention is that AAV can be produced in a cell transfection system without the incorporation of contaminant DNA upstream of the P5 promoter. Levels of contaminant DNA are a release criterion for AAV gene therapy trials, and reduction of contaminant DNA levels would improve both the purity of the vector, and the likelihood of a vector being cleared for trial.

Thus, the rAAV described herein find particular use in the treatment of subjects in need thereof, e.g., in the treatment of hemophilia and other monogenic disorders. In such applications, the rAAV may be provided in a pharmaceutical composition including the rAAV vector in admixture with a pharmaceutically acceptable excipient, diluent or carrier. Examples of suitable pharmaceutical carriers are well known in the art and include phosphate-buffered saline solutions, water, emulsions, such as oil/water emulsions, various types of wetting agents, sterile solutions and the like. Such carriers can be formulated by conventional methods and are administered to the subject at a therapeutically effective amount.

The following non-limiting examples are provided to further illustrate the present invention.

### Example 1: AAV2 P5 Promoter Modifications

It has been demonstrated that an exogenous spacer (HS5) between the REP binding site and a transcription start site-localized Ying-Yang 1 (YY1) binding site reduces DNA contamination without a concomitant reduction in viral titer (WO 2021/242664). Thus, it was posited that packaging of contaminant plasmid sequences is related to the P5 promoter structure and rep gene expression during production. As such, by manipulating critical elements of the P5 promoter and modulating rep expression, the encapsidation of contaminant sequences can be reduced.

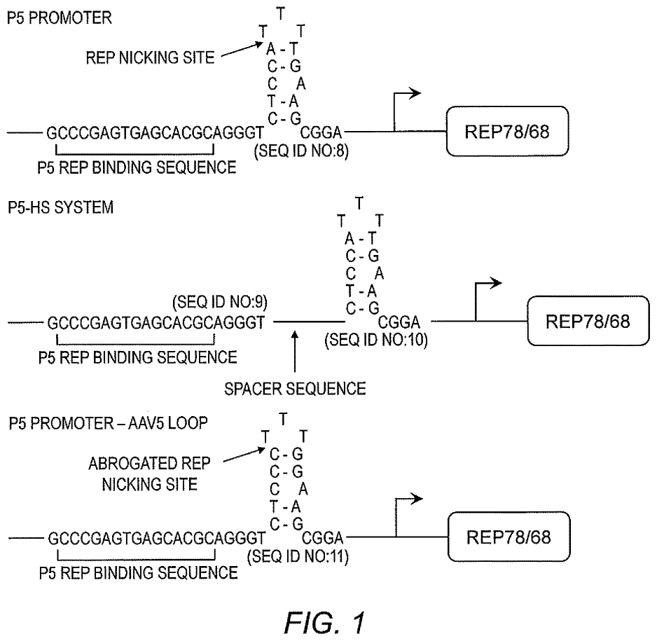

In one aspect, it was determined whether modifying the REP nicking site of the AAV2 P5 promoter would have an impact on titer and packaging of contaminant plasmid sequences. Accordingly, a series of rationally designed Terminal Resolution Sequence (TRS) loop modifications were generated (Table 1; FIG. 1).

TABLE 1

| | | | SEQ ID |
|---|---|---|---|
| Ex. | AAV2 P5 Promoter | TRS Loop Sequence | NO: |
| 1 | WT AAV2 (CR21) | ctccATtttgaag | 1 |
| 2 | CR21 + HS5 | ctccATtttgaag | 1 |
| 3 | AAV2 P5 + AAV5 TRS Loop | ctccCTttggaag | 2 |
| 4 | TRS dinucleotide GC | ctccGCtttgaag | 3 |
| 5 | TRS dinucleotide GT | ctccGTtttgaag | 4 |
| 6 | TRS dinucleotide TT | ctccTTtttgaag | 5 |
| 7 | TRS dinucleotide AA | ctccAAtttgaag | 6 |
| 8 | TRS dinucleotide CA | ctccCAtttgaag | 7 |

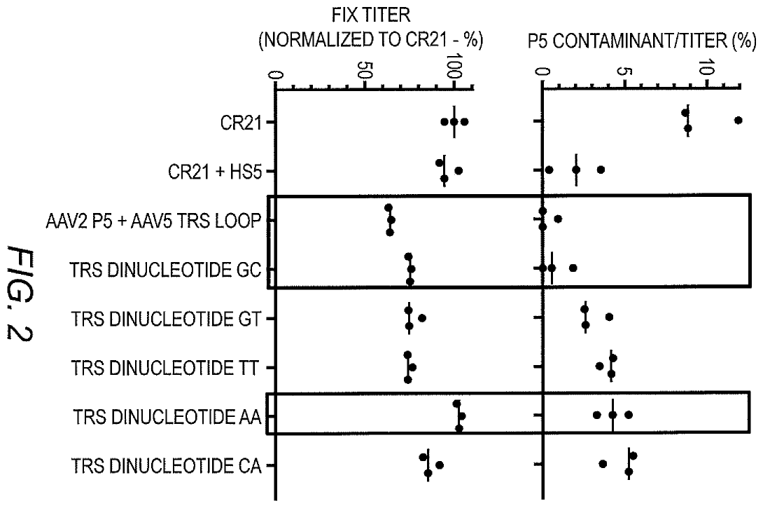

The results of this analysis indicated that, similar to the introduction of a spacer between the REP binding site and the YY1 binding site, the use of rationally designed TRS loop modifications to the P5 promoter can reduce encapsidation of nearby DNA by approximately ten- to 100-fold (FIG. 2).

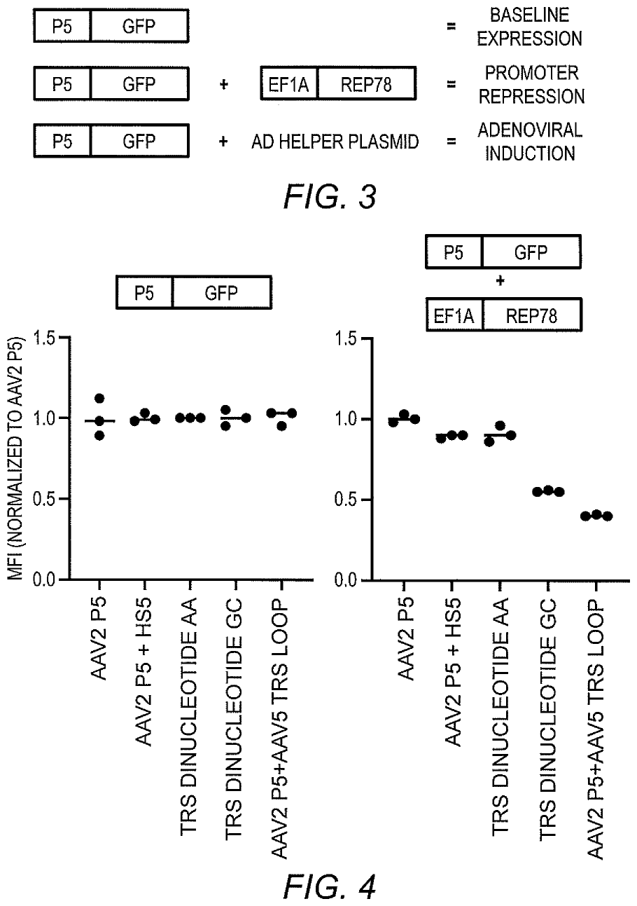

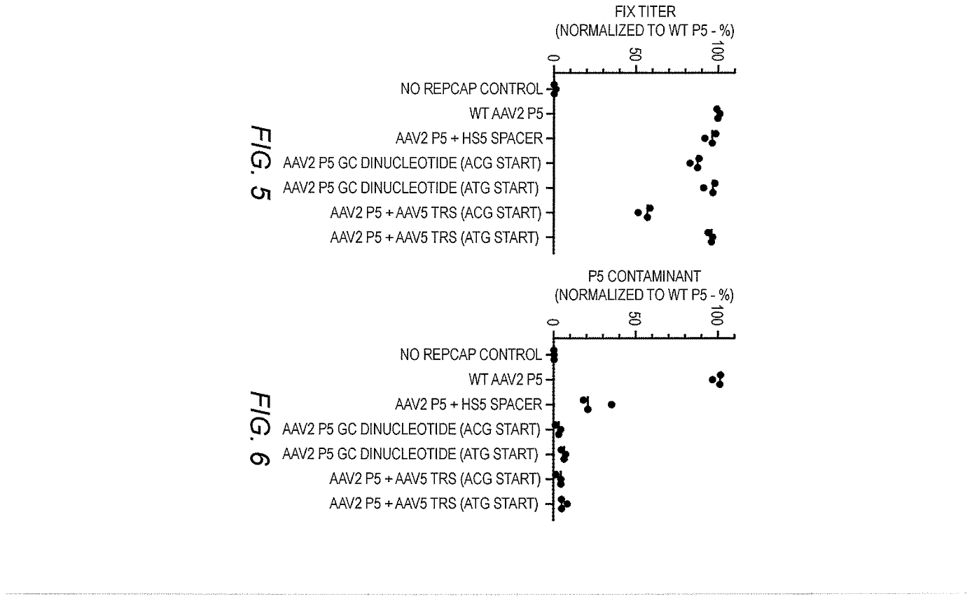

To quantify the impact of the AAV5 (Table 1, Ex. 3), GC dinucleotide (Table 1, Ex. 4), and AA dinucleotide (Table 1, Ex. 7) TRS loop modifications, a green fluorescent protein reporter assay was developed (FIG. 3). This assay indicated that TRS alterations that significantly reduce contamination also impact promoter function (FIG. 4). It was subsequently determined whether modifying the REP start codon from ACG to ATG could compensate for this observed reduction in promoter activity. This analysis indicated that changing the start codon compensated for losses in viral titer when an AAV5 (Table 1, Ex. 3) or GC dinucleotide (Table 1, Ex. 4) TRS loop was used in the P5 promoter (FIG. 5) without altering encapsidation of contaminants (FIG. 6).

It was subsequently determined whether the entire P5 promoter could be replaced. Thus, the use of P5 homologues from alternative serotypes were examined as replacements for the P5 promoter in rAAV production. In particular, the following P5 homologues were analyzed: AAV1 P5 homologue, AAV4 P5 homologue, AAV8 P5 homologue, AAV12 P5 homologue, and AAV13 P5 homologue, (Table 2). AAV1, AAV4, and AAV8 P5 homologues were as described in the literature, i.e., unmodified. The nucleotide sequences for the AAV12 and AAV13 P5 promoter homologues were obtained from GENBANK. However, the 5′ sequence was missing from these two promoters. Therefore, the first 5′ nucleotides were grafted from the 5′ region of the AAV2 P5 promoter.

TABLE 2

| | | SEQ ID |
|---|---|---|
| Promoter | Sequence | NO: |
| AAV1 P5 | cgcgaagcgcctcccacgctgccgcgtcagcgctgac | 13 |
| Homologue | gtaaattacgtcataggggagtggtcctgtattagct | |
| (P1) | gtcacgtgagtgcttttgcgacattttgcgacaccac | |
| | gtggccatttagggtatatatggccgagtgagcgagc | |
| | aggatctccattttgaccgcgaaatttgaacgagcag | |
| | cagcc | |
| AAV4 P5 | ctgacgtcaatgtgacgtcctagggttagggaggtcc | 14 |
| Homologue | ctgtattagcagtcacgtgagtgtcgtatttcgcgga | |
| (P4) | gcgtagcggagcgcataccaagctgccacgtcacagc | |
| | cacgtggtccgtttgcgacagtttgcgacaccatgtg | |
| | gtcaggagggtatataaccgcgagtgagccagcgagg | |
| | agctccattttgcccgcgaattttgaacgagcagcag | |
| | cc | |
| AAV8 P5 | gcgcgaagcgcctcccacgctgccgcgtcagcgctga | 15 |
| Homologue | cgtaaattacgtcataggggagtggtcctgtattagc | |
| (P8) | tgtcacgtgagtgcttttgcggcattttgcgacacca | |
| | cgtggccatttgaggtatatatggccgagtgagcgag | |
| | caggatctccattttgaccgcgaaatttgaacgagca | |
| | gcagcc | |
| AAV12 P5 | *CATAGAGGTCCTGTATTAGAGGTCACGTGAGTGTTT | 16 |
| Homologue | TGCGACAGTTTGCGACACCATGTGGTCACaagaggta | |
| (P12) | tataaccgcgagtgagccagcgaggagctccattttg | |
| | cccgcgaagtttgaacgagcagcagcc | |
| AAV13 P5 | *CATAGAGGTCCTGTATTAGAGGTCACGTGAGTGTTT | 17 |
| Homologue | TGCGACATTTTGCGACACCATGTGGTCACgctgggta | |
| (P13) | tttaaccgcgagtgagcgaaccaggagctccattttg | |
| | cccgcgaattttgaacgagcagcagcc | |

*Uppercase sequences: grafted from AAV2 P5 promoter.

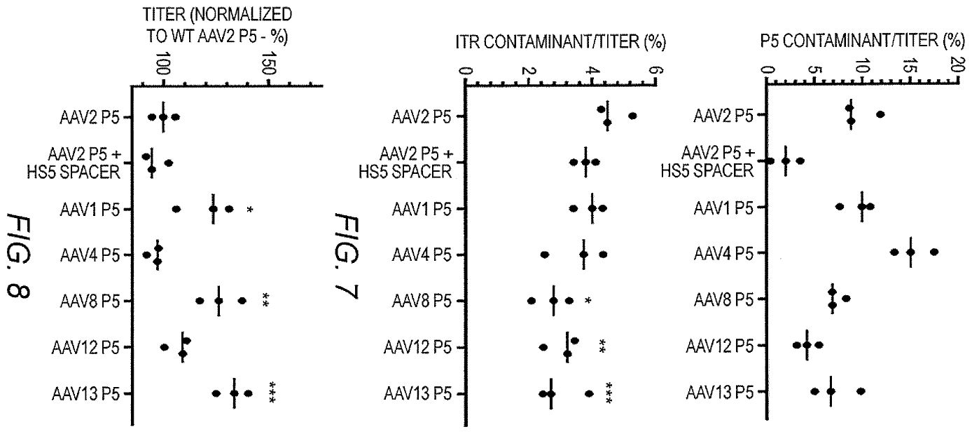

It was observed that replacing the canonical AAV2 P5 promoter with alternative homologues reduced the frequency of encapsidation of contaminants from distant hotspots by over 20% (FIG. 7). A subset of these surrogate promoters also improved viral titer by 30-40% during small scale rAAV production in a transgene agnostic manner (FIG. 8). However, while AAV2 P5 tolerates TRS modifications (FIG. 2), similar modifications in the surrogate promoters did not tolerate such modifications (FIG. 9), suggesting differential promoter regulation.

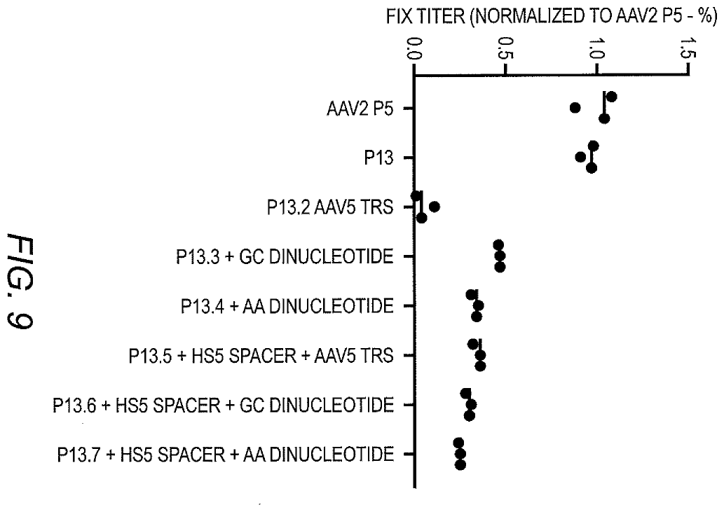

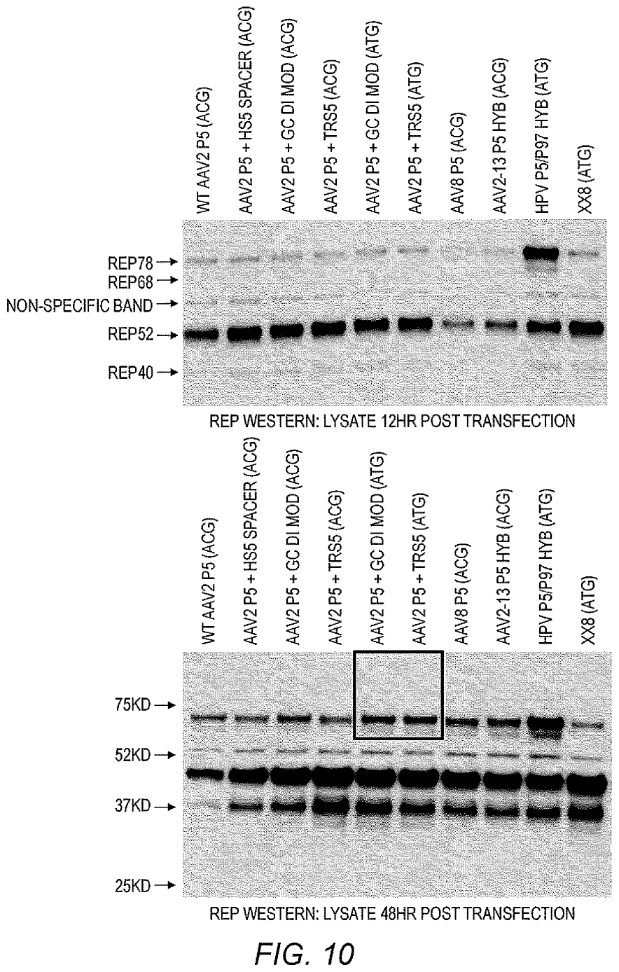

To further examine the implications of promoter modifications on promoter function REP western blot analyses were conducted (FIG. 10) These analyses indicated that modifications that improved purity and had the most detrimental effect on viral titer, had the strongest impact on promoter activity. Specifically, decreased large REP expression and increased small REP expression was observed (FIG. 10). These data indicate that Rep binds to mutant P5 tighter or for longer resulting in the enhanced transactivation of P19. Notably, replacing the P5 promoter with a non-related viral promoter (HPV P5), results in aberrant REP expression, low titers, and high contamination. Moreover, the use of ATG notably increased Rep78 expression after 48 hours (FIG. 10).

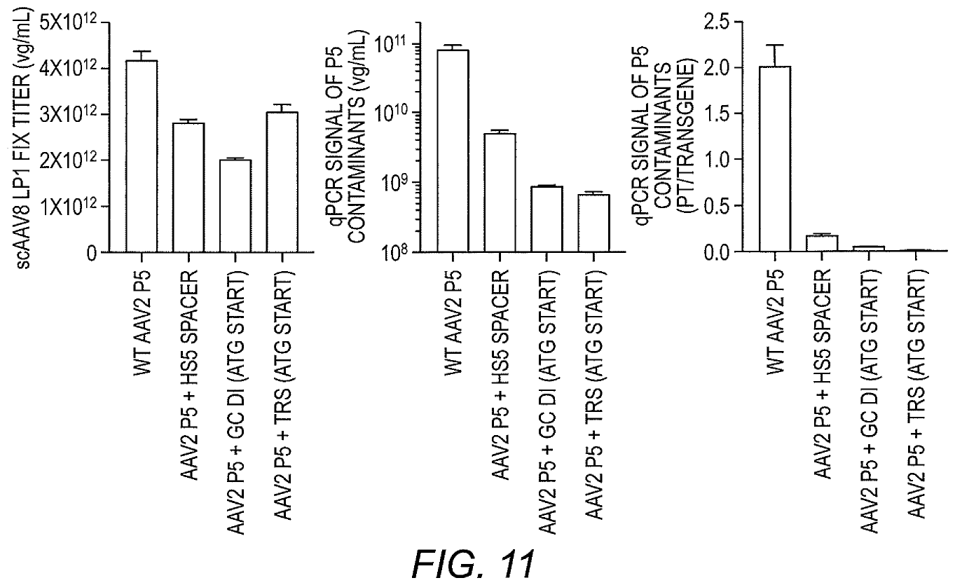

The preparation of vectors was scaled up to further demonstrate a quantitative reduction in DNA contamination. As shown in FIG. 11, there was a 2-log reduction in contamination (center panel). When the signal was represented as a proportion of the titer, this contamination was reduced from 2% to 0.2% with the spacer and to less than 0.05% in the TRS-modified constructs (FIG. 11, right panel).

The results of these analyses indicated that mutating the REP78 nick site on AAV2 P5 promoter effectively blocks replication originating at this site. Nick site modifications reduce the basal level of expression of REP78 and increase small REP expression. However, compensating for loss in large REP expression rescues vector titer thereby enabling efficient production of rAAV. In addition, the use of P5 homologues can reduce contamination outside of ITRs by reducing large REP expression. Accordingly, one or more of the above-referenced modified promoters provide viable alternatives to the canonical AAV2 P5 routinely used in rAAV production.

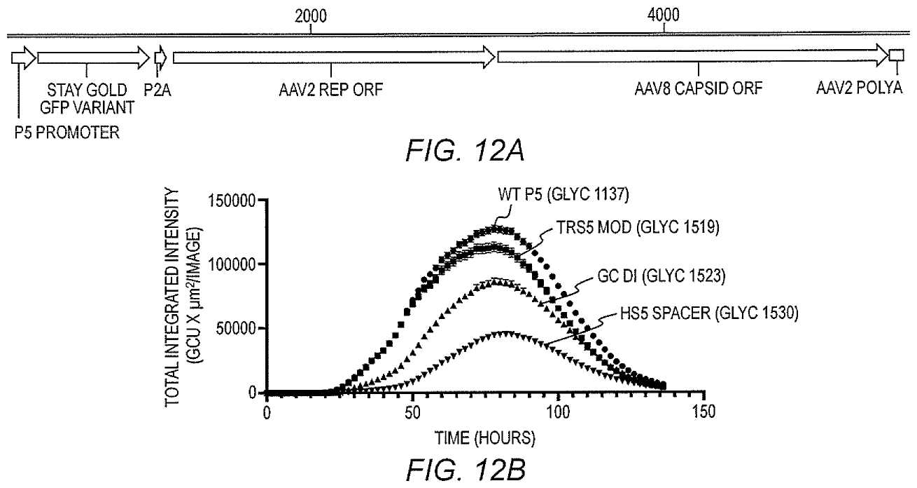

A plasmid was designed so that Rep78 and green fluorescent protein (GFP) were coexpressed during AAV production (FIG. 12A). Promoter activity was quantified in an IncuCyte® Live-Cell Analysis System every two hours. Different versions of this construct were generated including all of the different P5 modifications that showed the desired activity. Consistent with western blot analysis, live cell imaging showed that P5 activity was reduced with all of the P5 modifications (FIG. 12B).

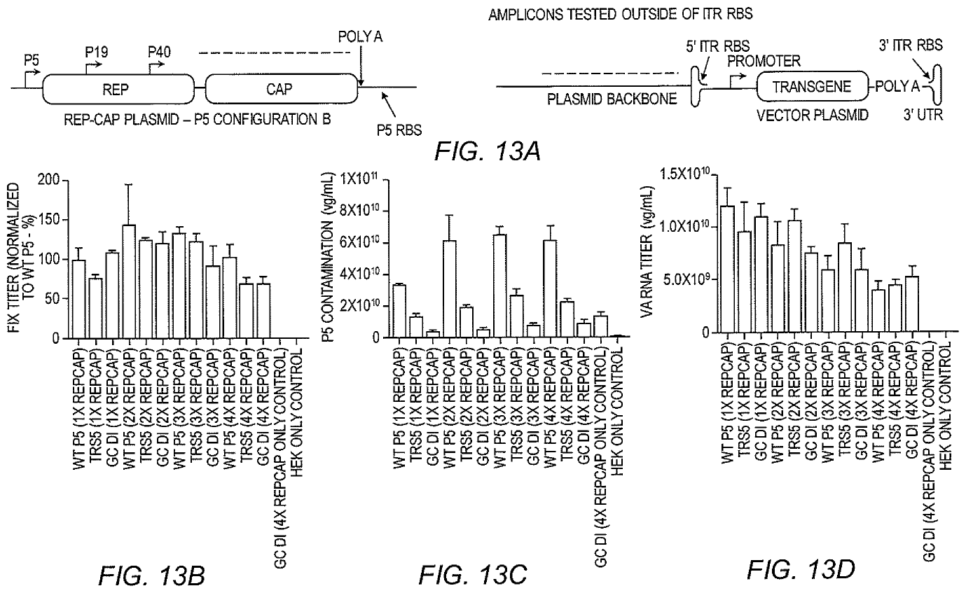

The ratio between the transgene/adenoviral helper plasmid and REP-CAP plasmid was optimized to about 1:1 for efficient production. To determine whether the reduction in expression observed by western blot and live imaging analysis was associated with the lower titers and whether improving expression would improve titer, higher amounts of REP-CAP plasmid were used. For this analysis, the quantity of transgene/adenoviral helper plasmid was kept constant and the amount of REP-CAP plasmid was increased 1×, 2×, 3× and 4× so that the ratios between these two plasmids were 1:1, 1:2, 1:3, and 1:4, respectively. The results of this analysis indicated that titer improved (FIG. 13B) and purity was maintained (FIGS. 13A, 13C-3D). Initial experiments were carried out with 6-well plates, the wells of which were approximately 2-3 cm in diameter. When scaled up to 15-cm plates, similar results were obtained. It was noted that the 4× amount of REP-CAP plasmid decreased titer likely because there was too much REP expression or the amount of the transfection reagent (polyethylenamine) needed to transfect elevated levels of DNA was toxic. Additional experiments were conducted to reduce the plasmid ratios to 1:0.75, 1:0.5, and 1:0.25. This analysis resulted in a reduction in titer.

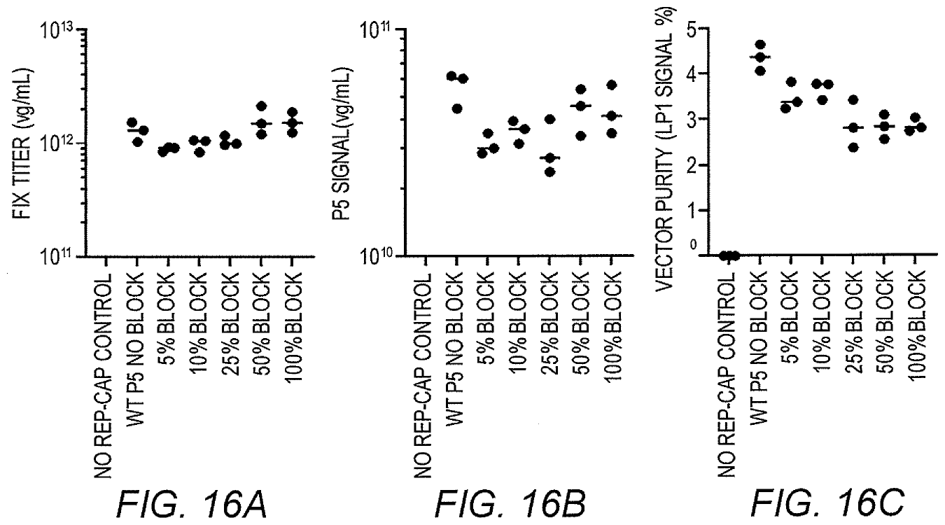

Most notably, the P5 signal (contaminant signal) was substantially increased when more wild-type P5 was added, but not when mutant P5 was added (FIGS. 13A, 13C). In addition, the presence of another contaminant (outside of the ITRs; FIG. 13A, 13D) was reduced as well. Not to be bound by theory, it is believed that this is due to competitive inhibition. By adding increased amounts of RBEs that cannot be packaged efficiently, the extra REP is sequestered thereby preventing it from packaging contaminant DNA. To demonstrate competitive inhibition (FIG. 16A-16C), HEK cells were transfected to initiate AAV production and a double-stranded oligonucleotide was included that sequestered excess REP. The oligonucleotide contained the sequence of a RBE, the GCTC repeat, with a 5′ and 3′ chemical modification to prevent incorporation into the vector, or extension by any polymerase. The two oligos (/5InvddT/GCTCGCTCGCTCGCTCGCTCGCTC/3InvdT/, SEQ ID NO:20; and /5InvddT/GAGCGAGCGAGCGAGCGAGCGAGC/3InvdT/, SEQ ID NO:21) were mixed, annealed and then mixed in with the AAV production plasmids at a 5%, 10%, 25%, 50% or 100% molar ratio to the amount of REP-CAP plasmid used in production.

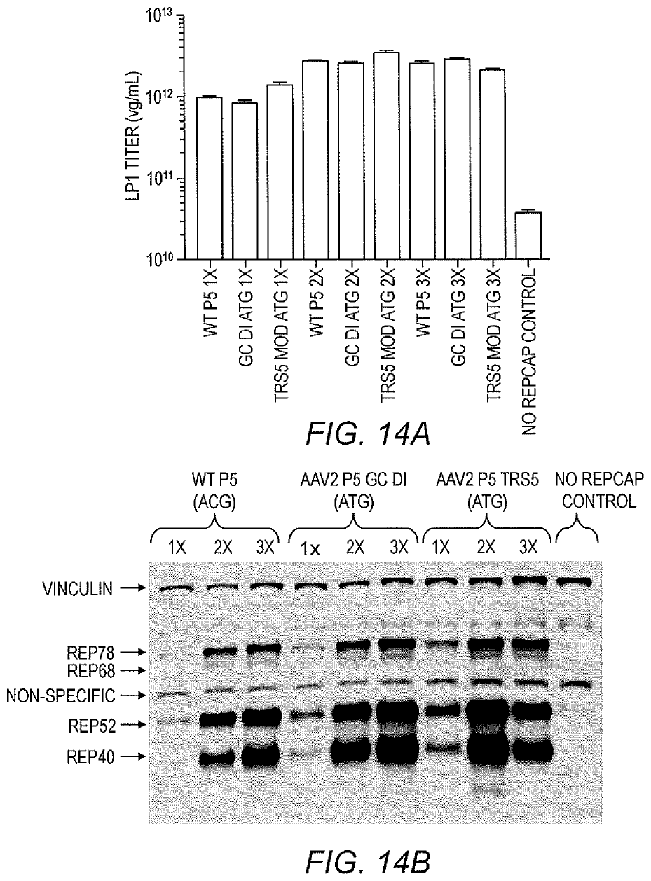

Western blot analysis was carried out to assess REP expression of samples prepared on 15-cm plates. This analysis indicated that as the amount of REP-CAP plasmid transfected increased, titer (FIG. 14A) and REP expression (FIG. 14B) also increased. It was surprising how much more REP expression was needed to obtain comparable titers when the P5 promoter was mutated. It is also surprising that excess REP expression did not lead to more contamination.

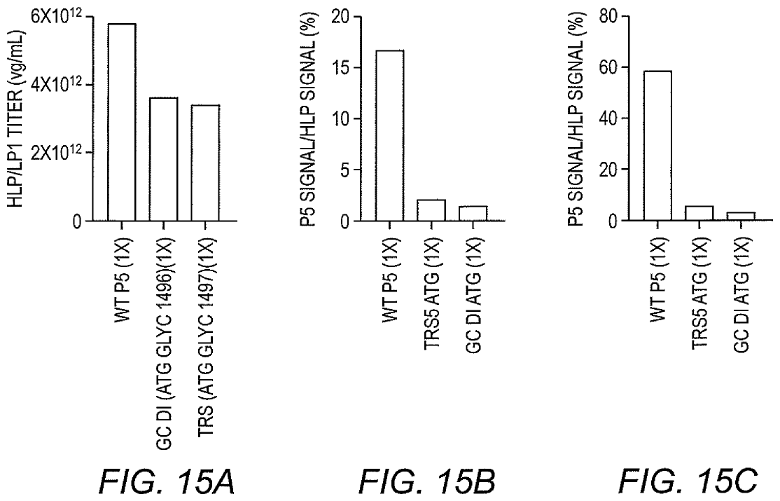

An expression cassette was prepared to assess whether the inclusion of a transgene had an impact on contamination. FVIII was selected as the transgene because it has been previously shown that FVIII is oversized and usually has higher rates of contamination than the FIX transgene. This analysis showed that approximately 75% of the AAV8 titer was obtained using a modified P5 as compared to wild-type P5 (FIG. 15A). Notably, the improvements to purity were maintained when either a 1:1 (1×, FIG. 15B) or 1:3 (3×, FIG. 15B) ratio of transgene/adenoviral helper plasmid:REP-CAP plasmid was used.

The FIX and FVIII vectors described in FIG. 11 and FIG. 15 and summarized in Table 3 were sequenced via Illumina sequencing to get an unbiased wholistic account of what is packaged, instead of qPCR snapshots. Table 4 provides data demonstrating that the overall vector purity improves as indicated by the percentage of reads that align to the transgene.

TABLE 3

| NGS Sample | Transgene | Rep-Cap |
|---|---|---|
| 1 | ssAAV8 HLP VIII | WT p5 |
| 2 | ssAAV8 HLP VIII | AAV2 P5 GC DI ATG |
| 3 | ssAAV8 HLP VIII | AAV2 P5 TRS5 ATG |
| 4 | ssAAV8 Lp1 FIX | WT p5 |
| 5 | ssAAV8 Lp1 FIX | AAV2 P5 GC DI ATG |
| 6 | ssAAV8 Lp1 FIX | AAV2 P5 TRS5 ATG |

TABLE 4

| | | % Reads | & Reads | % Reads not |
|---|---|---|---|---|
| | % Reads | aligning | aligning | aligning to |
| NGS | aligning to | to plasmid | to HEK | production |
| Sample | transgene | backbone | genome | system |
| 1 | 92.03 | 7.40 | 0.10 | 0.47 |
| 2 | 93.99 | 5.55 | 0.11 | 0.35 |
| 3 | 94.64 | 4.87 | 0.10 | 0.39 |
| 4 | 97.77 | 1.81 | 0.04 | 0.38 |
| 5 | 98.73 | 0.90 | 0.05 | 0.32 |
| 6 | 98.63 | 0.98 | 0.05 | 0.33 |

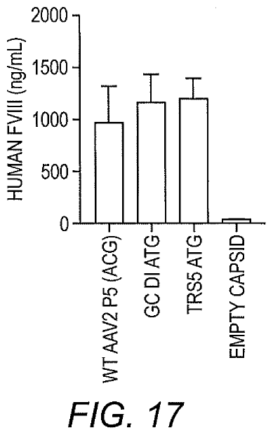

To assess expression in vivo, C57bl6 mice were given 2e10vg of ssAAV8 HLP FVIII vector (as in Tables 3 and 4 and FIGS. 15A-15C) via teil vein injection. At 7 days post vector administration, plasma was collected and analyzed for human FVIII expression. While the mean expression trends were higher, there was no significant difference between the samples (FIG. 17).

What is claimed is:

1. A recombinant adeno-associated virus (AAV) vector comprising a polynucleotide encoding AAV2 Replication (REP) 68/78 proteins operably linked to
(a) a P5 promoter homologue; or
(b) a P5 promoter comprising a terminal resolution sequence having a nucleotide sequence of ctccnntttgaag (SEQ ID NO:18), wherein “nn” is any dinucleotide except “at;” or
(c) a P5 promoter comprising a terminal resolution sequence having a nucleotide sequence of ctccn₁tttn₂gaag (SEQ ID NO:19), wherein each of n₁ and n₂ is independently an a, c, t or g, with the proviso that when n₁ is “a,” n₂ is not “t.”

2. The AAV vector of claim 1, wherein the P5 promoter homologue comprises a nucleotide sequence of SEQ ID NO:13-17.

3. The AAV vector of claim 1, wherein the polynucleotide encoding AAV2 REP68/78 proteins comprises a start codon having the nucleotide sequence of ATG or ACG.

4. A host cell comprising the AAV vector of claim 1.

5. A recombinant P5 promoter comprising a terminal resolution sequence having a nucleotide sequence of
(a) ctccnntttgaag (SEQ ID NO:18), wherein “nn” is any dinucleotide except “at;” or
(b) ctccn₁tttn₂gaag (SEQ ID NO:19), wherein each of n₁ and n₂ is independently an a, c, t or g, with the proviso that when n₁ is “a,” n₂ is not “t.”

6. An adeno-associated virus (AAV) vector comprising the recombinant P5 promoter of claim 1.

7. The AAV vector of claim 6, wherein said AAV is selected from the group of AAV1, AAV2, AAV3, AAV4, AAV5, AAV6, AAV7, AAV8, AAV9, AAV10, AAV11, AAV12, AAV13, and a synthetic variant thereof.

8. The AAV vector of claim 6, further comprising a polynucleotide encoding AAV2 Replication (REP) 68/78 proteins operably linked to the recombinant P5 promoter.

9. The AAV vector of claim 8, wherein the REP68/78 start codon has the nucleotide sequence of ATG or ACG.

10. A host cell comprising the recombinant P5 promoter of claim 5.

11. A host cell comprising the AAV vector of claim 6.

12. A method for increasing titer and/or purity of a recombinant adeno-associated virus (rAAV) comprising operably linking a polynucleotide encoding AAV2 Replication (REP) 68/78 proteins to
(a) a P5 promoter homologue;
(b) a P5 promoter comprising a terminal resolution sequence having a nucleotide sequence of ctccnntttgaag (SEQ ID NO:18), wherein “nn” is any dinucleotide except “at;” or
(c) a P5 promoter comprising a terminal resolution sequence having a nucleotide sequence of ctccn₁tttn₂gaag (SEQ ID NO:19), wherein each of n₁ and n₂ is independently an a, c, t or g, with the proviso that when n₁ is “a,” n₂ is not “t,”
thereby increasing the titer and/or purity of the rAAV.

13. The method of claim 12, wherein the P5 promoter homologue comprises a nucleotide sequence of SEQ ID NO:13-17.

14. The method of claim 12, wherein the polynucleotide encoding AAV2 REP68/78 proteins comprises a start codon having the nucleotide sequence of ATG or ACG.
